(19) United States
(12) Patent Application Publication
(10) Pub. No.: US 2002/0138093 A1
(43) Pub. Date: Sep. 26, 2002

(54) Intra-aortic balloon catheter having a gas lumen insert

(76) Inventors: Philip Song, Passaic, NJ (US); Boris Leschinsky, Waldwick, NJ (US); Olga Laksin, Scotch Plains, NJ (US); George Peters, Wayne, NJ (US)

Correspondence Address: Abraham Ronai, Datascope Corp., 14 Philips Parkway, Montvale, NJ, 07645, US

(21) Appl. No.: 09/813,905

(22) Filed: Mar. 21, 2001

(51) Int. Cl. A61M 29/00

(52) U.S. Cl. 606/194; 606/191

(57) ABSTRACT

An intra-aortic balloon catheter having a gas lumen insert disposed within its gas lumen during insertion of the catheter into the blood vessel of a patient. The gas lumen insert facilitates insertion and helps prevent kinking of the catheter.

### BACKGROUND OF THE INVENTION

1. Field of the Invention

The invention relates to an intra-aortic balloon (IAB) catheter. More particularly, the invention relates to an IAB catheter having a gas lumen insert for enhanced insertability.

2. Description of the Prior Art

IAB catheters are used in patients with left heart failure to augment the pumping action of the heart. The catheters, approximately one (1) meter long, have an inflatable and deflatable balloon at the distal end. The catheter is typically inserted into the femoral artery and moved up the descending thoracic aorta until the distal tip of the balloon is positioned just below or distal to the left subclavian artery. The proximal end of the catheter remains outside of the patient's body. A passageway for inflating and deflating the balloon extends through the catheter and is connected at its proximal end to an external pump. The patient's central aortic pressure is used to time the balloon and the patient's ECG may be used to trigger balloon inflation in synchronous counterpulsation to the patient's heart beat.

IAB therapy increases coronary artery perfusion, decreases the workload of the left ventricle, and allows healing of the injured myocardium. Ideally, the balloon should be inflating immediately after the aortic valve closes and deflating just prior to the onset of systole. When properly coordinated, the inflation of the balloon raises the patient's diastolic pressure, increasing the oxygen supply to the myocardium; and balloon deflation just prior to the onset of systole lowers the patient's diastolic pressure, reducing myocardial oxygen demand.

IAB catheters may also have a secondary passageway or lumen which can be used to measure aortic pressure. In this dual lumen or co-lumen construction, the secondary lumen may also be used to accommodate a guide wire to facilitate placement of the catheter and to infuse fluids, or to do blood sampling.

Typical dual lumen IAB catheters have an outer, flexible, plastic tube, which serves as the inflating and deflating gas passageway, and a central tube therethrough formed of plastic tubing, stainless steel tubing, or wire coil embedded in plastic tubing. A polyurethane compound is used to form the balloon. Other IAB catheters on the market have a co-lumen configuration wherein the inner tube is connected to or embedded in the outer tube wall.

A great deal of effort has been exerted in an effort to reduce the outer diameter of the dual or co-lumen IAB catheter. A reduction in size is desired in order to minimize the size of the arterial opening, to facilitate percutaneous insertion of the catheter into the aorta, maximizing blood flow past the inserted catheter, and also to allow for the use of a smaller insertion sheath to further maximize distal flow. Progress has certainly been made: IAB catheters currently on the market have outer diameters of as low as 8.0 Fr compared to over 10.0 Fr only a few years ago. Progress has been incremental, however, because of the difficulties encountered in reducing component sizes while still maintaining the necessary physical design requirements of the overall catheter required for efficient counterpulsation therapy and for smooth percutaneous insertion into the patient's blood vessel.

A reduction in the outer diameter or profile of the IAB catheter results in a catheter having a lower overall stiffness. The movement towards low profile dual or co-lumen IAB catheters has resulted in the production of catheters that tend to be more difficult to advance into the femoral artery and catheters that tend to kink more often during percutaneous insertion. Simply increasing the size of the catheter to increase stiffness or using a different material for the catheter body are not acceptable options given the amount of design work already dedicated to creating an IAB catheter that performs counterpulsation therapy in a clinically desirable manner.

Datascope Corp. sells a single lumen pediatric IAB having a removable metal stylet in its gas passageway for catheter stiffness enhancement. The stylet occupies less than a third of the cross sectional area of the IAB gas lumen, thus not providing significant protection against the type of kinking induced via a percutaneous insertion. In contrast to the dual or co-lumen IABs which are inserted percutaneously, the pediatric IAB is inserted through a surgical cut down procedure.

### SUMMARY OF THE INVENTION

Accordingly, it is an object of the invention to produce a device to enhance the insertion properties of a percutaneously insertable IAB which does not change the physical design characteristics of the IAB during counterpulsation therapy.

It is another object of the invention to produce a small profile intra-aortic balloon catheter with good insertion properties.

It is yet another object of the invention to produce a small profile intra-aortic balloon catheter that does not kink during insertion.

The invention is an improved intra-aortic balloon catheter having a removable gas lumen insert. The gas lumen insert comprises an elongated body preferably having approximately the same cross sectional shape as the gas lumen of the catheter. The IAB catheter is inserted into the blood vessel of a patient with the gas lumen insert disposed within the inner lumen of the catheter, thereby enhancing the stiffness and insertability of the catheter. Prior to initiation of therapy the insert is removed from within the catheter and the patient.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is longitudinal cross section of a prior art dual lumen intra-aortic balloon catheter.

FIG. 1A is a transverse cross section of the prior art intra-aortic balloon catheter taken along line 1A-1A.

FIG. 2 is longitudinal cross section of a dual lumen intra-aortic balloon catheter incorporating the gas lumen insert of the present invention.

FIG. 2A is a transverse cross section of the dual lumen intra-aortic balloon catheter of FIG. 2 taken along line 2A-2A.

FIG. 3 is longitudinal cross section of a prior art co-lumen intra-aortic balloon catheter.

FIG. 3A is a transverse cross section of the prior art co-lumen intra-aortic balloon catheter taken along line 3A-3A.

FIG. 4 is longitudinal cross section of a co-lumen intra-aortic balloon catheter incorporating the gas lumen insert of the present invention.

FIG. 4A is a transverse cross section of the co-lumen intra-aortic balloon catheter of FIG. 2 taken along line 4A-4A.

### DETAILED DESCRIPTION OF THE PREFERRED EMBODIMENTS

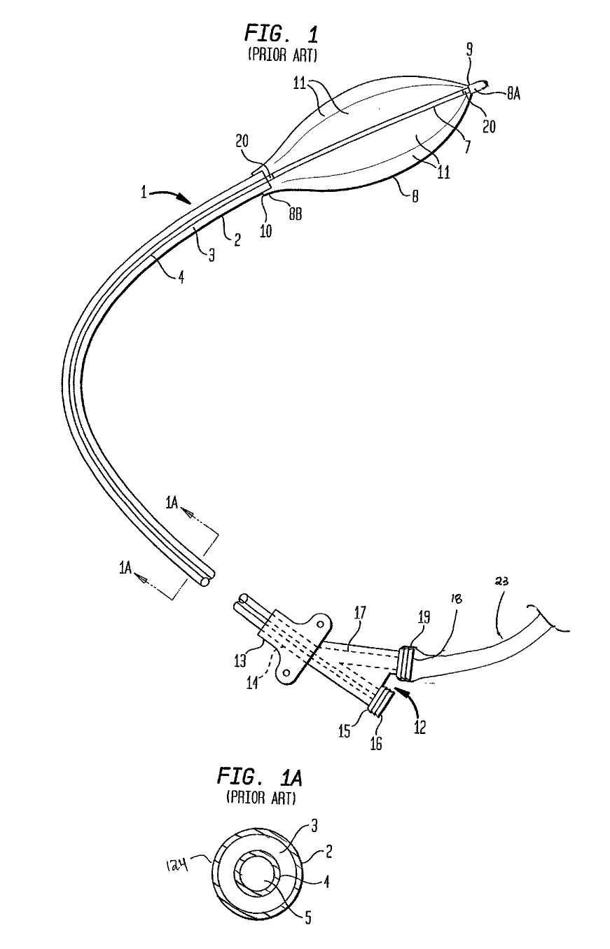

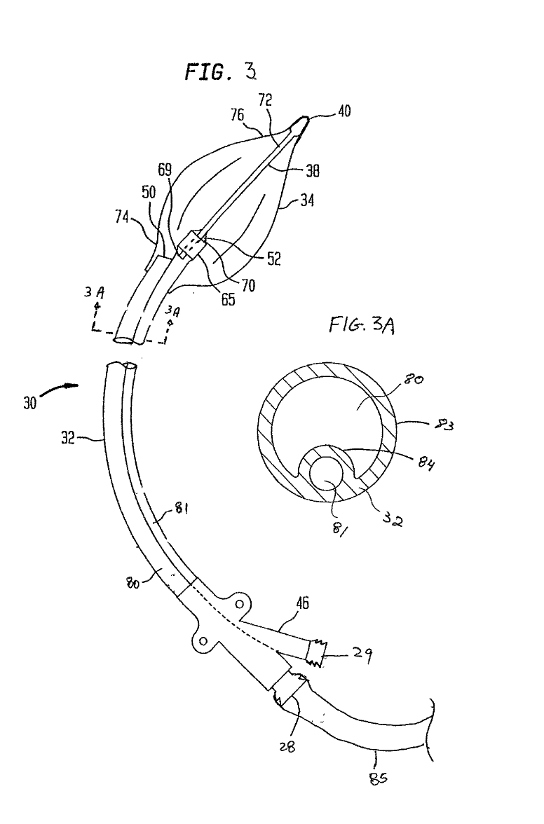

Three general prior art intra-aortic balloon catheter structures are currently on the market, a dual lumen configuration, a co-lumen configuration, and to a much more limited extent a single lumen configuration. The general structure of a dual lumen IAB catheter is best described in relation to FIGS. 1 and 1A which illustrate a dual-lumen prior art intra-aortic balloon catheter, generally designated 1. The general structure of a co-lumen IAB catheter, generally designated 30, is best described in relation to FIGS. 3 and 3A.

Turning to FIGS. 1 and 1A first, catheter 1 is constructed of an outer tube 2 forming a gas lumen 3; and a central tube 4 disposed within outer tube 2 and creating a central lumen 5 as may best be seen in FIG. 1A. Outer tube 2 is preferably made at least partially from polyurethane but may also be made from nylons, polyetheramide or polyesteramide (PEBAX materials, manufactured by Elf-Atochem), or other similar materials. Central tube 4 is preferably made at least partially from polyurethane, including but not limited to Estane, made by B. F. Goodrich, Tecoflex or Tecothane made by Thermedics, and Pellethane made by Dow, but may also be made from polyimide, nylon, Pebax or a Nitinol alloy. Nitinol is a kink-resistant superelastic shape memory metal alloy manufactured and sold by, among others, Rayehem Corp. Central lumen 5 and gas lumen 3 are disposed within an outer surface 124 of outer tube 2.

A balloon 8 is disposed at the distal end of the catheter 1. Distal portion 7 of central tube 4 extends beyond distal end 10 of outer tube 2. Distal end 8A of balloon 8 is attached to a tip 9 formed on distal end 7 of central tube 4. Proximal end 8B of balloon 8 is attached, for example by means of a lap joint, to distal end 10 of outer tube 2. Distal portion 7 of central tube 4 supports balloon 8. Said distal portion 7 must have sufficient strength to prevent inversion of balloon 8 as it inflates and deflates under aortic pressure, but at the same time, be flexible enough to be safely inserted through an introducer sheath, moved through the arterial tree, and maintained in the thoracic aorta.

Balloon 8 is formed of a nonthrombogenic flexible material and may have folds 11 formed as a result of wrapping balloon 8 about central tube 4 to ease insertion of catheter 1. Balloon 8 has a single wall thickness of between one (1) to five (5) mils. Balloon 8 is preferably stretch blow molded polyurethane, see co-pending U.S. patent application Ser. Nos. 09/545,763 and 09/757,859 herein incorporated by reference in their entirety, but may also be made from polyurethane. Radio-opaque band 20 at the distal end of catheter 1 aids in positioning balloon 8 in the descending aorta.

Inflation and deflation of balloon 8 is accomplished through gas lumen 3. Central lumen 5 can accommodate a guide wire for placement or repositioning of catheter 1. When the guide wire is not disposed in central lumen 5, central lumen 5 may be used for measuring blood pressure in the descending aorta. This pressure measurement may be used to coordinate the inflation and deflation of balloon 8 with the pumping of the heart, however, use of the patient's ECG is preferred. Additionally, central lumen 5 may be used to infuse liquids into the descending aorta, or to sample blood.

At proximal end 12 of catheter 1 a hub 13 is formed on proximal end 14 of outer tube 2. Central lumen 5 extends through hub 13 and a connector 16 is provided at proximal end 15 (or exit) of central lumen 5. Measurement of aortic pressure and blood sampling may be done through proximal end 15 of central lumen 5. Proximal end 18 of gas lumen 3 exits through a side arm 17 of hub 13 on which is provided a connector 19. Proximal end 18 of gas lumen 3 may be connected to an intra-aortic balloon pump via extracorporeal tubing 23.

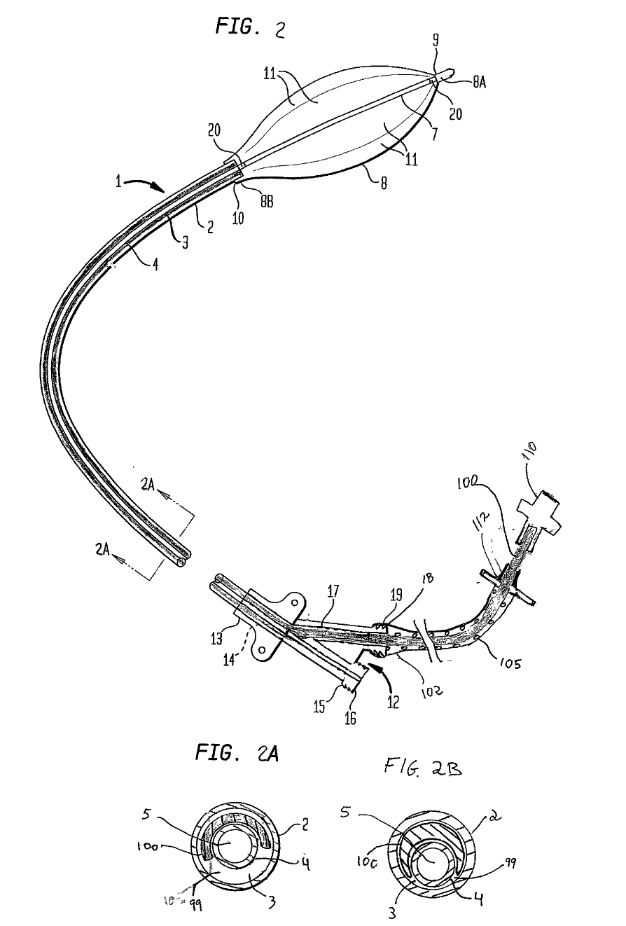

FIG. 2 illustrates the IAB catheter 1 of FIG. 1 with a gas lumen insert 100 disposed within the gas lumen 3 between an inner surface of the outer tube 2 and an outer surface of the inner tube 4, see FIG. 2A. Gas lumen insert 100 extends from the distal end 10 of outer tube 2 through connector 19 into extracorporeal tubing 102 and terminates in a one-way valve 110. A gap 99, as best seen in FIG. 2A, exists between the gas lumen insert 100 and the outer tube 2 allowing stagnant air to withdrawn from the balloon 8 through valve 110. A coil 105 is disposed between the gas lumen insert 100 and the extracorporeal tubing 102. Extracorporeal tubing 102 is preferably made from polyvinylchloride (PVC) or polyurethane, but may be made from any flexible clear polymer as well. Coil 105 reduces the friction between extracorporeal tubing 102 and gas lumen insert 100 and facilitates removal of gas lumen insert 100 from catheter 1. Coil 105 is preferably made from steel but may be made from any appropriate polymer or other metal.

Gas lumen insert 100, as illustrated in FIGS. 2, 2A, and 2B, is generally a half circle but may alternatively wrap further around inner tube 4, to enhance stiffness or for other design reasons, so long as inner tube 4 is not hindered from exiting through proximal end 15 of the central lumen 5. Gas lumen insert 100 is preferably made from PEBAX or nylon, however, other materials including but not limited to metals and plastics may be used. In order to facilitate withdrawal of gas lumen insert 100, a lubricant is superficially applied along the length of gas lumen insert 100. The material and geometry of the gas lumen insert 100 may vary depending the amount of additional stiffness desired to add to catheter 1.

In order to allow for a gas lumen insert having a maximum cross sectional area, inner tube 4 may be shifted to an off center position, as illustrated in FIG. 2B. In a single lumen IAB, the gas lumen is preferably designed to occupy substantially the entire gas lumen and to allow for an air gap to remove stale air from the balloon 8.

As illustrated in FIG. 2, gas lumen insert 100 extends to the end of outer tube 2. In an alternate embodiment of catheter 1, inner tube 4 may comprise two parts connected end-to-end, a first part at least partially disposed within outer tube 2 and a second part disposed within balloon 8. The gas lumen insert 100 used with this alternate catheter should preferably extend beyond the end of outer tube 2 so as to overlap the joint between the first and second parts of the inner tube, thus preventing a stress concentration point at the joint.

IAB catheter 1 is inserted into the blood vessel of a patient with gas lumen insert 100 inside gas lumen 3. Luer fitting 112 at the proximal end of extracorporeal tubing 102 is connected to valve 110 during insertion. Gas lumen insert 100 enhances the stiffness of catheter 1, facilitating insertion and preventing kinking during insertion. A guide wire is inserted into the blood vessel of a patient through an angiographic needle or another means known in the art. Catheter 1 is then advanced over the guide wire into the blood vessel with or without the use of an insertion sheath. If an insertion sheath is used, it is advanced into the blood vessel over the guide wire. Catheter 1 is then advanced over the guide wire through the sheath into the blood vessel to a position appropriate for pumping. Gas lumen insert 100 is removed from catheter 1 prior to the initiation of therapy by pulling valve 110 and gas lumen insert 100 proximally while holding catheter 1 still.

FIG. 3 illustrates a longitudinal cross section of prior art co-lumen catheter 30 comprising a co-lumen tube 32 connected on its proximal end to a y-fitting connector 46 and on a distal end to a proximal end 74 of a balloon 34. Co-lumen tube 32, as best seen in FIG. 3A, has an outer tube portion 83, defining a gas lumen 80, and an inner tube portion 84, defining an inner lumen 81, embedded in the wall of the co-lumen tube 32. Inner lumen 81 is disposed within an outer surface 120 of co-lumen tube 32 and gas lumen 80 is disposed within a first inner surface 122 of co-lumen tube 32. A distal end 52 of inner tube portion 84 extends beyond a distal end 50 of outer tube portion 83 and is connected to a proximal end 69 of an inner lumen extension tube 38 via a crimp 65. A distal end 72 of inner lumen extension tube 38 is connected to a tip 40 and to a distal end 76 of balloon 34. Inner lumen extension tube 38 is preferably made from Nitinol, polyimide, nylon, polyether-ether-ketones (PEEK), and other appropriate materials. Co-lumen tube 32 is preferably made from polyurethane but may also be made from Pebax.

Note that co-lumen tube 32 may also comprise a smaller tube or channel affixed along its length to the inside surface of a larger tube. Note further that in an alternate embodiment of catheter 30 inner lumen extension tube 38 may be replaced with a tube, preferably made from polyimide, that is disposed within the length of inner lumen 81 and extends into the balloon 34 all the way to tip 40, see U.S. Pat. No. 6,024,693, herein incorporated by reference in its entirety.

Balloon 34 is formed of a nonthrombogenic flexible material and may have folds formed as a result of wrapping balloon 34 about inner lumen extension tube 38 to ease insertion of the catheter 30. Balloon 34 has a single wall thickness of between one (1) to five (5) mils. Balloon 34 is preferably stretch blow molded polyurethane, see co-pending U.S. patent application Ser. Nos. 09/545,763 and 09/757,859 herein incorporated by reference in their entirety, but may also be made at least partially from through a regular solvent casting process.

Inner lumen 31 terminates in port 29 of y-fitting connector 46. Measurement of aortic pressure and blood sampling may be done through port 29. A proximal end of gas lumen 80 exits through a port 28 on y-fitting connector 46. A proximal end of gas lumen 80 may be connected to an intra-aortic balloon pump via extracorporeal tubing 85.

Extracorporeal tubing 85 is preferably made from polyvinylchloride (PVC) but may be made from polyurethane or any clear and flexible polymer, as well. The details of the co-lumen catheter construction are more fully laid out in U.S. Pat. No. 6,024,693, herein incorporated by reference in its entirety.

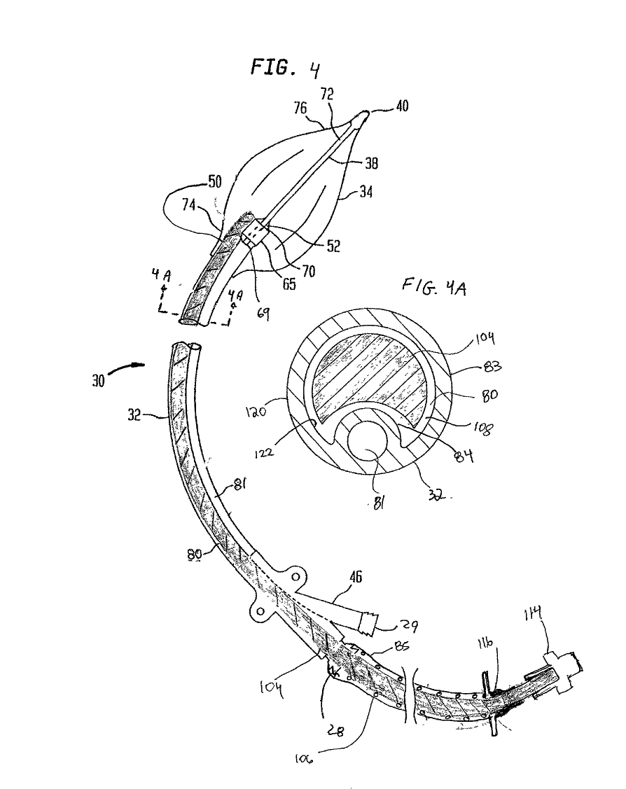

FIGS. 4 and 4A illustrate the catheter 30 of FIG. 3 with a gas lumen insert 104 in gas lumen 80. Gas lumen insert 104 preferably extends just beyond a distal end 50 of outer tube portion 83 so as to overlap a stress concentration point created by the connection between inner tube portion 84 and inner lumen extension tube 38. An air gap 108 exists between gas lumen insert 104 and outer tube portion 83 and inner tube portion 84 which allows for withdrawal of stagnant air from balloon 34 through one-way valve 114.

Gas lumen insert 104 is preferably made from polyether amide (Pebax), however, other materials including but not limited to metals and plastics may be used. The material and geometry of gas lumen insert 104 may vary depending the amount of additional stiffness desired to add to catheter 30. In order to facilitate withdrawal of gas lumen insert 104, a lubricant is superficially applied along the length of gas lumen insert 104. Furthermore, a coil 106 is disposed within extracorporeal tubing 85, between an outer surface of gas lumen insert 104 and an inner surface of extracorporeal tubing 85, so as to prevent sticking of the gas lumen insert 104 to the wall of the extracorporeal tubing 85.

Catheter 30 is inserted into the blood vessel of a patient with the gas lumen insert 104 disposed within catheter 30 similar to dual lumen catheter 1 detailed above. Luer fitting 116 is connected to valve 114 during insertion. Gas lumen insert 104 enhances the stiffness of catheter 30, facilitating insertion and preventing kinking during insertion. Gas lumen insert 100 is removed from catheter 30 prior to the initiation of therapy by pulling valve 114 and gas lumen insert 104 proximally while holding catheter 30 still.

As many apparently widely different embodiments of the present invention can be made without departing from the spirit and scope thereof, it is to be understood that the invention is not limited to the specific embodiments thereof except as defined in the appended claims.

What is claimed is:

1. A percutaneously insertable intra-aortic balloon catheter comprising a catheter tube, a balloon membrane, a tip, and a gas lumen insert, said catheter tube comprising a gas lumen disposed within an outer surface of the catheter tube, a proximal end of the balloon membrane is connected to a distal end of the catheter tube, a distal end of the balloon membrane is connected to the tip, the gas lumen insert comprising a removable elongate body at least partially disposed within the gas lumen.

2. A percutaneously insertable intra-aortic balloon catheter comprising a catheter tube, a balloon membrane, a tip, and a gas lumen insert, said catheter tube comprising an inner lumen and a gas lumen disposed within an outer surface of the catheter tube, a proximal end of the balloon membrane is connected to a distal end of the catheter tube, a distal end of the balloon membrane is connected to the tip, the gas lumen insert comprising a removable elongate body at least partially disposed within the gas lumen.

3. A percutaneously insertable intra-aortic balloon catheter comprising a catheter tube, a balloon membrane, a tip, and a gas lumen insert, said catheter tube comprising an inner tube portion, defining an inner lumen, and an outer tube portion, defining a gas lumen, a distal portion of said inner tube portion extending beyond a distal end of the outer tube portion and being connected to a distal end of the balloon membrane and to the tip, the gas lumen insert comprising a removable elongate body at least partially disposed within the gas lumen.

4. A percutaneously insertable intra-aortic balloon catheter comprising a catheter tube, a balloon membrane, an inner tube, a tip, and a gas lumen insert, said catheter tube comprising an inner tube portion and an outer tube portion defining a gas lumen, said inner tube being at least partially disposed within the inner tube portion and extending beyond a distal end of the outer tube portion and being connected to a distal end of the balloon membrane and to the tip, the gas lumen insert comprising a removable elongate body at least partially disposed within the gas lumen.

5. A percutaneously insertable intra-aortic balloon catheter comprising an outer tube, an inner tube, a balloon membrane, a tip, and a gas lumen insert, said inner tube being disposed within the outer tube, a distal portion of said inner tube extending beyond a distal end of the outer tube and being connected to a distal end of the balloon membrane and the tip, the gas lumen insert comprising a removable elongate body at least partially disposed within the gas lumen.

6. The intra-aortic balloon catheter as claimed in claim 1 or 2 or 3 or 4 or 5 wherein the gas lumen insert is at least partially coated with a lubricant.

7. The intra-aortic balloon catheter as claimed in claim 1 or 2 or 3 or 4 or 5 further comprising a coil and a connector, said connector being connected to a proximal end of the catheter and having a gas lumen port and an inner lumen port, said gas lumen port communicating with said gas lumen and said inner lumen port communicating with said inner lumen, said gas lumen port being connected to a distal end of an extracorporeal tube, the gas lumen insert passing through said gas lumen port and said extracoporeal tubing, said coil being disposed in the extracorporeal tubing between an inner surface of the extracorporeal tubing and an outer surface of the gas lumen insert.

8. The intra-aortic balloon catheter as claimed in claim 1 or 2 or 3 or 4 or 5 further comprising a coil and a connector, said connector being connected to a proximal end of the catheter and having a gas lumen port and an inner lumen port, said gas lumen port communicating with said gas lumen and said inner lumen port communicating with said inner lumen, said gas lumen port being connected to a distal end of an extracorporeal tube, the gas lumen insert passing through said gas lumen port and said extracoporeal tubing, said coil being disposed in the extracorporeal tubing between an inner surface of the extracorporeal tubing and an outer surface of the gas lumen insert, said gas lumen insert terminating in a one-way valve, said extracorporeal tubing terminating in a connector for connection to said one-way valve.

9. The intra-aortic balloon catheter as claimed in claim 3 wherein the distal portion of the inner tube portion is made from a different material than the portion of the inner tube portion disposed within an outer surface of the catheter.

10. The intra-aortic balloon catheter as claimed in claim 3 wherein the distal portion of the inner tube portion is made from a different material than the portion of the inner tube portion disposed within an outer surface of the catheter, and wherein the distal portion of the inner tube portion and a distal end of the catheter are connected at a joint, the gas lumen insert extends beyond the distal end of the outer tube portion and overlaps the joint.

11. The intra-aortic balloon catheter as claimed in claim 5 wherein the distal portion of the inner tube is made from a different material than the portion of the inner tube disposed within an outer surface of the catheter.

12. The intra-aortic balloon catheter as claimed in claim 5 wherein the distal portion of the inner tube is made from a different material than the portion of the inner tube disposed within an outer surface of the catheter, and wherein the distal portion of the inner tube and a distal end of the catheter are connected at a joint, the gas lumen insert extends beyond the distal end of the outer tube and overlaps the joint.

13. A percutaneously insertable intra-aortic balloon catheter comprising a catheter tube, a balloon membrane, an inner lumen extension tube, a tip, a connector, a coil, an extracorporeal tube, and a gas lumen insert,
said catheter tube comprising an inner tube portion, defining an inner lumen, and an outer tube portion, defining a gas lumen, a proximal end of said inner lumen extension tube being connected to a distal end of the inner tube portion at a joint, a distal end of said inner lumen extension tube being connected to a distal end of the balloon membrane and to the tip,
the gas lumen insert comprising a removable elongate body at least partially disposed within the gas lumen, said gas lumen insert extending beyond the distal end of the outer tube portion and overlapping the joint,
the connector being connected to a proximal end of the catheter and having a gas lumen port and an inner lumen port, said gas lumen port communicating with said gas lumen and said inner lumen port communicating with said inner lumen, said gas lumen port being connected to a distal end of the extracorporeal tube, the gas lumen insert passing through said gas lumen port and said extracoporeal tubing, said coil being disposed in the extracorporeal tubing between an inner surface of the extracorporeal tubing and an outer surface of the gas lumen insert, said gas lumen insert terminating on its proximal end in a one-way valve, said extracorporeal tubing terminating on its proximal end in a connector for connection to said one-way valve.

14. The intra-aortic balloon catheter as claimed in claim 13 wherein the catheter tube is at least partially made from polyurethane and the gas lumen insert is at least partially made from polyether block amide.

15. The intra-aortic balloon catheter as claimed in claim 2 or 3 or 4 wherein the catheter tube is at least partially made from polyurethane and the gas lumen insert is at least partially made from polyether block amide.

16. The intra-aortic balloon catheter as claimed in claim 5 wherein the outer tube is at least partially made from polyurethane and the gas lumen insert is at least partially made from polyether block amide.

17. The intra-aortic balloon catheter as claimed in claim 1 or 2 or 3 or 4 or 5 wherein the gas lumen occupies more than one third of the cross sectional area of the gas lumen.

18. The intra-aortic balloon catheter as claimed in claim 1 or 2 or 3 or 4 or 5 where in the gas lumen occupies at least one half of the cross sectional area of the gas lumen.

19. A method for insertion of an intra-aortic balloon catheter comprising a catheter tube, a balloon membrane, a tip, and a gas lumen insert, said catheter tube comprising a gas lumen disposed within an outer surface of the catheter tube, a proximal end of the balloon membrane is connected to a distal end of the catheter tube, a distal end of the balloon membrane is connected to the tip, the gas lumen insert comprising a removable elongate body at least partially disposed within the gas lumen, comprising the steps of:
a) Percutaneously inserting the catheter into a blood vessel of a patient;
b) Advancing the catheter into the blood vessel to a position appropriate for therapy; and
c) Removing the gas lumen insert from within the gas lumen by pulling the gas lumen proximal the catheter.

20. A method for insertion of an intra-aortic balloon catheter comprising a catheter tube, a balloon membrane, a tip, and a gas lumen insert, said catheter tube comprising an inner lumen and a gas lumen disposed within an outer surface of the catheter tube, a proximal end of the balloon membrane is connected to a distal end of the catheter tube, a distal end of the balloon membrane is connected to the tip, the gas lumen insert comprising a removable elongate body at least partially disposed within the gas lumen, comprising the steps of:
a) Percutaneously inserting the catheter into a blood vessel of a patient;
b) Advancing the catheter into the blood vessel to a position appropriate for therapy; and
c) Removing the gas lumen insert from within the gas lumen by pulling the gas lumen proximal the catheter.

21. The method as claimed in claim 19 or 20 wherein the gas lumen insert occupies more than one third of the cross sectional area of the gas lumen.

22. The method as claimed in claim 19 or 20 wherein the gas lumen insert occupies at least one half the cross sectional area of the gas lumen.
